(19) United States
(12) Patent Application Publication
(10) Pub. No.: US 2002/0136778 A1
(43) Pub. Date: Sep. 26, 2002

(54) Milk-enhancing feedstuff and method

(76) Inventors: Robert M. Claycamp, Seymour, IN (US); Robin L. Hayes, Rensselaer, IN (US)

Correspondence Address: Holiday W. Banta, Esquire, Woodard, Emhardt, Naughton, Moriarty & McNett, Bank One Center/Tower, 111 Monument Circle, Suite 3700, Indianapolis, IN, 46204-5137, US

(21) Appl. No.: 09/757,961

(22) Filed: Jan. 10, 2001

(51) Int. Cl. A61K 35/54; A01N 63/00; A01N 63/04; A61K 35/78

(52) U.S. Cl. 424/581; 424/93.51; 424/750

(57) ABSTRACT

Improved feed compositions for lactating ruminants are provided that employ a high quality inedible egg product to increase the butterfat content and the milk protein content of the ruminant's milk. These improved compositions include one or more high quality inedible egg products in an amount from about 1% to 100% of the total weight of the composition. The balance of these improved compositions may consist of any ingredient(s) in any combination when such composition is capable of meeting or exceeding the nutritional requirements of the species to be fed. One aspect of the present invention provides a method for producing a high quality inedible egg product. In another aspect of the invention, there are provided feedstuff compositions for lactating ruminants that may be adapted to various stages of lactation. This invention also provides an improved diet program wherein a lactating ruminant is fed various embodiments of a feed composition according to the present invention depending upon the ruminant's stage of lactation. This improved diet program allows a herdsman to optimize butterfat and milk protein production throughout the lactation cycle with no deleterious effects in terms of the health or milk production of the ruminant. Animals fed these improved compositions benefit from a significant increase in the butterfat and milk protein composition of their milk compared to lactating ruminants fed prior art diets.

### FIELD OF THE INVENTION

The invention relates generally to synthesized feedstuffs for ruminants, and more particularly to feed compositions containing high quality inedible egg products as the main ingredient and to a method of producing and feeding the same.

### BACKGROUND OF THE INVENTION

In the field of milk production for commercial uses, farmers are paid for the milk that their ruminant animals produce in part based upon the butterfat and milk protein content of that milk. It is therefore important to such farmers to maximize the butterfat content or the milk protein content, or both, of their animals' milk. Previous efforts to increase butterfat and/or milk protein content have generally comprised increasing the levels in ruminant diets of coarse fiber ingredients, or buffer supplements such as sodium bicarbonate and magnesium oxide when coarse fiber feeds are not available. This is because high fiber rations produce high levels of acetic acid in the rumen, acetic acid being the primary precursor to butterfat.

Prior efforts to increase butterfat percentage have also comprised using expensive, highly processed feed products that increase the herdsman's production costs and thereby diminish the return yielded by the higher market price for the improved butterfat percentage milk. These prior efforts also have yielded only small improvements in butterfat and/or milk protein percentage and may introduce undesirable byproducts into the ruminant's milk. This latter consideration is important to herdsmen in today's market because modem consumers are becoming increasingly conscious of and concerned by any unnatural byproducts contained in the milk they purchase.

Some examples of such prior efforts include the addition of high protein sources to the ruminant's diet such as soybean meal, rumen-protected biologically active substances such as amino acids, and alkali treated proteinaceous feed supplements. Examples of these types of compositions can be found in U.S. Pat. No. 4,225,620 to Rawlings et al., U.S. Pat. No. 5,236,717 to Vinci, U.S. Pat. No. 5,244,681 to Vinci et al., U.S. Pat. No. 5,250,307 to Cummings et al., and U.S. Pat. No. 5,871,773 to Rode et al.

These supplemental ingredients are highly perishable, as a general rule, which requires the farmer to purchase small quantities frequently or invest in refrigerated storage facilities. These drawbacks further increase the cost of feeding such supplements because farmers must invest significant time and energy both maintaining adequate fresh supplies of the supplements and measuring and mixing the supplement materials with the ruminants' regular rations.

These prior art compositions therefore leave significant room for improvement in terms of composition, manufacturing method, and feeding method. The present invention is addressed to such improvement.

### SUMMARY OF THE INVENTION

The present invention relates to egg-based feed compositions for lactating ruminants that consist essentially of a high quality inedible egg product or products in an amount from about 1% to 100% by weight and comprise at least one ingredient selected from the group consisting of milk products, cereal grain or cereal grain products, fruit pectin, other carbohydrates, fiber, fat, urea, electrolytes, vitamins, minerals, yeast, and other animal protein and/or vegetable protein as the balance of the composition. The feed composition may be pelletized or unpelletized.

The provision of high quality inedible egg in the combinations of the present invention imparts a desirable amino acid profile to the feedstuffs and yields a highly palatable ruminant feed. When fed to ruminant animals, these feed compositions are capable of increasing the butterfat percentage of the animals' milk. These feed compositions are also capable of increasing the milk protein percentage of the animals' milk. Further, when the instant compositions comprise a complete feed, with or without a forage or silage element, substantial labor savings in terms of feed preparation may be enjoyed thereby.

The present invention also relates to a method of increasing butterfat percentage and milk protein percentage in lactating ruminants. The method comprises the steps of preparing an egg-based feed composition for a lactating ruminant producing milk with a butterfat percentage that consists essentially of a high quality inedible egg product or products in an amount from about 1% to 100% by weight and comprise at least one ingredient selected from the group consisting of milk products, cereal grain or cereal grain products, fruit pectin, other carbohydrates, fiber, fat, urea, electrolytes, vitamins, minerals, yeast, and other animal protein and/or vegetable protein as the balance of the composition; and feeding the egg-based feed composition to the lactating ruminant to thereby obtain a second butterfat percentage of said milk that is higher than said butterfat percentage.

Additionally, the present invention includes a method of preparing a high quality inedible egg product for use in feed products for lactating ruminants. This method comprises the steps of providing at least one inedible egg; denaturing said at least one inedible egg; refrigerating said at least one inedible egg; and pasteurizing said at least one inedible egg.

These and other objects, advantages, and features are accomplished according to the compositions and methods disclosed in the following description of the preferred embodiments of the present invention.

### DESCRIPTION OF THE PREFERRED EMBODIMENTS

For the purposes of promoting an understanding of the principles of the invention, reference will now be made to the preferred embodiments thereof, and specific language will be used to describe the same. It will nevertheless be understood that no limitation of the scope of the invention is thereby intended, such alterations, modifications, and further applications of the principles of the invention being contemplated as would normally occur to one skilled in the art to which the invention relates.

Before proceeding further, those of ordinary skill in the art will recognize that feeding the feed compositions as hereinafter more fully described can improve the butterfat and milk protein content of any lactating ruminant's milk. It is therefore intended that the scope of the present invention include and comprise compositions adapted to meet the nutritional requirements of any such lactating ruminant. This is so regardless of the fact that the majority of the following description discusses the invention in terms of particular formulations for and the benefits of feeding these formulations to dairy cows.

The present invention concerns the use of a high quality inedible egg product as a significant source of protein and other beneficial nutrients in a diet for lactating ruminants. Therefore, one aspect of the present invention concerns a method of manufacturing a high quality inedible egg product. The term “high quality inedible egg product” as used in the present disclosure refers to inedible eggs (“inedible eggs” defined below) that have been prepared according to a manufacturing method disclosed and taught as an aspect of the present invention. According to other aspects of the present invention, there are provided feedstuff compositions and diet programs for lactating ruminants that may be adapted to various stages of lactation.

The high quality inedible egg product that forms the basis of all the preferred diet compositions of the present invention begins with “inedible” eggs. Inedible eggs are defined to include those whole raw eggs, hard boiled eggs, egg yolks, egg albumen, and all other liquid or dry egg fractions that the United States Department of Agriculture (“USDA”) has mandated cannot be used in human feeds or for human consumption. Often the eggs that become inedible eggs are cracked, dirty, or misshapen. The term “inedible egg product” as used with this invention broadly encompasses any and all types of inedible eggs to which a dye has been added in conformance with USDA requirements.

The high quality of the inedible egg products contemplated for use with the present invention is obtained by employing the following method. Eggs destined to become inedible eggs are obtained, the shells broken, and a denaturant dye added to clearly show that the product is not for human consumption. Those of skill in the art will recognize those classes of compounds that may be used as the denaturant dye without affecting the nutritional analysis or makeup of the inedible egg. Next, the eggshells are removed by centrifuging the liquid egg through a filter, or by any other method known or contemplated for accomplishing the removal of shell debris from liquid egg.

The liquid egg obtained thereby is then held in refrigerated storage tanks at a temperature of about 45° F. or less until it can be delivered to the heat-pasteurizing unit, but preferably for not longer than about five days. Once the liquid egg is in the pasteurizer, it is heated to a temperature of at least 140° F. and held at that temperature for at least 4.5 minutes in order to kill all pathogenic and spoilage-inducing microorganisms. Again, those of skill in the art will recognize that the removal and/or growth inhibition of pathogenic and spoilage-inducing microorganisms may be accomplished by methods other than heat treatment, and such methods are intended and contemplated to come within the meaning of the term “pasteurizing” as it is used with the present invention.

At this point in the present manufacturing method, the manufacturer decides whether the inedible egg product will remain liquid or will be dried to produce a powder. If it is to remain liquid, the egg product is either packaged immediately and refrigerated or frozen or returned to a refrigerated storage tank until it can be packaged in an airtight and watertight container and then refrigerated or frozen. If it is to become a dry inedible egg product, then the inedible egg product is either spray dried immediately or returned to a refrigerated storage tank until it can be spray dried.

The inedible egg product may then be spray dried without any additives according to those procedures known in the art of spray drying. Upon completion of the spray drying process, a free-flow or anti-caking agent may be added to the inedible egg product to prevent the formation of hard lumps in storage. Preferably, the moisture level of the resultant dry inedible egg product is between about 2 and about 10 percent. More preferably, the moisture level of the resultant dry inedible egg product is between about 3 and about 7 percent; and most preferably, the moisture level of the resultant dry inedible egg product is between about 4 and about 6 percent.

Those of skill in the art will recognize that the use of a spray dryer is particularly desirable in that it minimizes damage to the proteins contained in the inedible egg product and therefore yields a very high quality inedible egg product. This dry inedible egg product is shelf-stable and may be packaged in airtight and watertight containers for shipment and sale, without requiring refrigeration or freezing for storage.

In the most preferred embodiments of this invention, whole inedible eggs are used to manufacture the high quality inedible egg product in order to obtain the full nutritional and flavor values possessed by whole eggs. The present high quality inedible egg product may be provided in a liquid or a dried form. The liquid form is preferably refrigerated during storage, although those of skill in the art will recognize that room temperature storage may be accomplished by methods known in the art such as chemical preservation and/or canning. Also, the liquid form may be blended with other preferred ingredients for convenience in storage, preparation, and feeding. The dried form does not require refrigeration during dry storage. The dried form may be reconstituted with water or other suitable liquid ingredients to form a liquid supplement according to the present invention, and it may also be dry blended with other preferred ingredients for convenience in storage, preparation, and feeding of a complete feed according to the present invention.

A preferred feed supplement embodiment of the present invention comprises the high quality inedible egg product in an amount from about 1% to 100% by weight of the composition. A preferred concentrate feed embodiment of the present invention comprises the high quality inedible egg product in an amount from about 1% to 100% by weight of the composition. The high quality inedible egg ingredient is typically present in the feed in the range of about 25% to 100% by weight, and more preferably from about 36% to about 65% by weight.

The balance of the present inventive feed supplement and concentrate feed compositions, when those compositions comprise less than 100% high quality inedible egg product, may consist of any desired ingredients in any desired combination capable of supplying the nutritional requirements of a lactating ruminant animal, including, but not limited to, milk products, cereal grain or cereal grain products, fruit pectin, other carbohydrates, fiber, fat, urea, electrolytes, vitamins, minerals, yeast, and other animal protein and/or vegetable protein sources.

As herein employed, the term “milk products” refers to and includes fluids secreted by the mammary glands of lactating female animals for the nourishment of their young and all fractions thereof and products derived therefrom. By way of example, and not of limitation, some milk products contemplated by and intended to come within the scope of the present invention include skim milk, buttermilk, whole whey, delactosed whey, casein, milk albumin, whey protein concentrate, whey permeates, whey sweet water, raw milk, powdered milk, and curd.

The term “cereal grain or cereal grain products” as used herein refers to the seeds or fruits of various feed plants including the cereal grasses and other plants such as the soybean, and non-byproduct fractions and derivatives thereof. By way of example, and not of limitation, some cereal grains contemplated for use with the compositions of the present invention include oats, barley, wheat, corn, cottonseed, flax, hops, rice, rye, safflower seed, sunflower seed, almonds, walnuts, peanuts, buckwheat, and broomcorn. “Other carbohydrates” as used in the present description describes a class of compounds formed of carbon, hydrogen, and oxygen that is capable of providing an easily digested ready source of energy to an animal. The present invention contemplates the use of any compound meeting the above description that is suitable for use in an animal feed, including complex sugars derived from various sources; simple sugars such as dextrose, fructose, galactose, and glucose; molasses; and starch. The foregoing list is provided for purposes of example only, and no limitation of the scope of the present invention is intended thereby. The term “fiber” as used with the present invention refers to various carbohydrate moieties that are not degradable by animal digestive enzymes and that provide an animal with roughage that is beneficial for the animal's digestive tract. Traditional fiber sources include forage such as hay, orchard grass, alfalfa, fescue, clover, and timothy; and silage. Some fibers useful for practicing the present invention can be pectin-containing substances, brans, hulls, shells, or screenings of grains, seeds, or nuts. Screenings are obtained from cleaning grain and seeds, and include light and broken grains, agricultural seeds, weed seeds, hulls, chaff, straw, milldust, sand, and dirt. Some examples of other fiber sources include oat hulls, almond hulls, barley mill run, bean hulls, peanut skins, rice hulls, peanut hulls, nutshells, grape pomace, apple pomace, oat shorts, wheat shorts, wheat middlings, flax hulls, and soybean mill run, citrus pulp, beet pulp, potato pulp, fruit peel, pea fiber, plant root or tuber fiber products, carrageenans, and vegetable and/or microbially derived gums. The fiber sources may be treated, i.e., modified, or untreated. Again, these particular fiber sources are provided as examples only, and no limitation of the scope of the present invention is intended. “Fat” refers to and describes any animal-edible compound capable of supplying the animal with a substantial amount of energy upon digestion of the compound. Some examples of useful fats include edible fats and oils from animal and vegetable sources such as mono-, di-, or tri-glycerides of various fatty acids such as stearic, palmitic, oleic, linoleic, lauric, and others. Animal-edible fats and oils can also include complex lipids such as phospholipids including fatty acid esters of glycerol phosphate or lecithin. Other fats that may be used include the oils, tailings or residues of soybean oil, corn oil, tallow, fish oil, coconut oil, palm oil, reclaimed restaurant fats and greases, acidulated soap stocks, and acidulated fats and oils. The foregoing list is provided for purposes of example only, and no limitation of the scope of the present invention is intended thereby.

The term “electrolytes” as used herein refers to a class of chemicals that will provide ionic conductivity when dissolved in water or when placed in contact with water. Suitable examples of this class of compounds include sodium chloride, sodium bicarbonate, and calcium carbonate. These particular electrolyte sources are provided as examples only, and no limitation of the scope of the present invention is intended.

The term “vitamin” as used herein refers to and includes all those organic compounds that are known to be essential to and/or used by animals to help regulate their metabolic processes but do not provide energy or serve as building units. As examples, the better known members of this class include vitamin A, vitamin B, vitamin B complex, vitamin C, vitamin D, vitamin E, vitamin K, and vitamin P. Many premixes of essential vitamins are available commercially for use in feeds for various animal species, including Microvit™ sold by Rhone-Poulenc Animal Nutrition and Custom Premixes sold by Animal Science Products. These particular vitamins and vitamin sources are provided as examples only, and no limitation of the scope of the present invention is intended. “Minerals” when used herein describes and includes those inorganic compounds, whether natural or synthesized, that are required by animals to carry out and/or regulate their metabolic processes. A common term used to refer to minerals generally that is intended to come within the foregoing definition is “trace elements.” Various examples of important mineral sources for animals include manganese, iodine, zinc, copper, cobalt, and iron compounds, and mineral salts such as dicalcium and tricalcium phosphate and monoammonium phosphate. Those of skill in the art of animal nutrition will recognize that, although the precise desired mineral source for a particular application is not listed above, the present invention nonetheless contemplates the inclusion of such desired mineral source. “Yeast” as used herein refers to any animal-edible unicellular organism belonging to the family Saccharomycetaceae. One particularly popular source of yeast for animal feeds is dried brewer's yeast. Again, the present invention contemplates and intends that all organisms, however prepared, meeting the foregoing definition come within the scope of this invention.

The present description uses the terms “other animal protein and/or vegetable protein sources” to mean and include animal-edible nitrogen sources. Examples of suitable protein ingredients include dried blood and meat meal from rendering plants, cottonseed meal, soybean meal, rapeseed meal, canola meal, sunflower meal, dehydrated alfalfa, dried and sterilized animal and poultry manure, fish meal, fish solubles, cell cream, corn gluten meal, feather meal, dried brewer's grains, and distiller's dried grains. As with all the foregoing definitions, the present recitation of examples is for the skilled artisan's reference only, and no limitation on the scope of the present invention is intended by the inclusion of these specific examples.

Preferably, the balance of the present feed supplement compositions comprises other carbohydrates and other animal protein and/or vegetable protein sources. It is also preferred that the balance of the present concentrate feed compositions comprises cereal grain or cereal grain products, other carbohydrates, fiber, other animal protein and/or vegetable protein sources, vitamins, and minerals.

In the most preferred embodiments of the present invention, the concentrate feed embodiments are tailored specifically to the needs of the particular species of ruminant animal to be fed and may be pelletized for greater nutrient availability and palatability. The preferred embodiments of the feed supplements do not have to be tailored to the particular species, and therefore they may be formulated to have the same or highly similar compositions across species lines.

While the foregoing constitutes a general description of the dietary compositions of the present invention, the following are specific examples of preferred compositions according to the present invention. The specific examples are provided for purposes of illustrating the invention and no limitations on the invention are intended thereby.

A preferred embodiment of the feed supplement composition of the present invention has the following formulation:

| | Percentage by Weight, |
|---|---|
| Ingredient | in the range of: |
| Dried Inedible Egg Product | From about 40 to about 60 |
| Distiller's Grains with Solubles | From about 35 to about 55 |
| Cane Molasses | From about 3 to about 7 |
| Total | 100 |

This formula may be processed into pellets or mixed for blending into other feeds.

A more preferred embodiment of the feed supplement composition of the present invention has the following formulation:

| Ingredient | Percentage by Weight |
|---|---|
| Dried Inedible Egg Product | 50 |
| Distiller's Grains with Solubles | 45 |
| Cane Molasses | 5 |
| Total | 100 |

A preferred embodiment of the concentrate feed composition of the present invention for use with lactating dairy cows has the following formulation:

| | Percentage by Weight, |
|---|---|
| Ingredient | in the range of: |
| Dried Inedible Egg Product | From about 40 to about 60 |
| Distiller's Grains with Solubles | From about 35 to about 55 |
| Cane Molasses | From about 3 to about 7 |
| Total | 100 |

As discussed above, it is generally quite important to a herdsman to increase the percentage of butterfat and/or milk proteins in his animal's milk, because he may thereby increase the market price of that milk. It is also important to minimize the amount of highly processed and/or man-made ingredients in ruminant feed, since modem consumers are becoming increasingly conscious of and concerned by any unnatural byproducts contained in their milk.

This invention also provides an improved diet program wherein a lactating ruminant is fed various embodiments of a feed composition according to the present invention depending upon the ruminant's stage of lactation. This improved diet program allows a herdsman to optimize butterfat and milk protein production throughout the lactation cycle with no deleterious effects in terms of the health or milk production of the ruminant. The preferred ration for lactating ruminants is adjusted regularly for maximum feeding efficiency. The production record of each ruminant is integrated into the calculation of energy and protein input to formulate the amount and quality of roughage and grains required to maintain the animal in top condition.

### EXPERIMENT

The following table demonstrates the ability of the feedstuff compositions of the present invention to significantly increase the butterfat and milk protein content of milk produced by animals fed these compositions over the butterfat and milk protein contents produced by animals fed a conventional diet. As used in the following table and description of the Experiment, the term “Egg” refers to and is intended to comprise the feed supplement composition embodiments of the present invention.

About 0.25 pound of Egg was added to the total mixed ration for each of 1,500 dairy cattle per day of the study. The total mixed ration was typical of current husbandry practices, being composed of roughage, such as hay, grass, or silage; grains, such as corn, wheat, milo, and/or fractions thereof; and minerals. The rations were balanced according to the following formulae in accordance with the diet program disclosed herein on a bi-weekly basis to maintain constant nutrient content throughout the entire study.

Nutritional Formula 1:
Target Values for High Production Group Cows

| | |
|---|---|
| Protein | 18.50% |
| Net Energy, Lactation | 0.80 Mcal/lb. |
| ADF (acid detergent fiber) | 19.00% |
| NDF (neutral detergent fiber) | 29.70% |
| Fat | 6.00% |
| Calcium | 1.10% |
| Phosphorus | 0.40% |

Nutritional Formula 2:
Target Values for Low Production Group Cows

| | |
|---|---|
| Protein | 17.50% |
| Net Energy, Lactation | 0.80 Mcal/lb. |
| ADF | 21.00% |
| NDF | 31.00% |
| Fat | 6.00% |
| Calcium | 1.05% |
| Phosphorus | 0.40% |

Production Results for Experiment

| | Daily | | Milk |
|---|---|---|---|
| Week of | Milk (lbs.) | Milk Fat % | Protein % |
| Before Egg Product | | | |
| March 31, 2000 | 84.5 | 3.59 | 3.00 |
| February 7 | nd | 3.54 | 3.03 |
| February 14 | 86.5 | 3.69 | 3.02 |
| February 28 | 84 | 3.60 | 2.97 |
| March 6 | 84 | 3.70 | 2.96 |
| March 13 | 86.3 | 3.61 | 2.97 |
| Average | 85.0 | 3.62 | 2.99 |
| Added 0.25 lb. Egg/day | | | |
| March 20 | 84.2 | 3.67 | 3.00 |
| March 27 | nd | 3.70 | 3.00 |
| April 3 | nd | 3.76 | 3.02 |
| April 10 | 87.1 | 3.81 | 3.02 |
| April 17 | 84.6 | 3.78 | 2.97 |
| Average | 85.3 | 3.74 | 3.00 |

nd = not determined

Experimental observations indicate that milk production levels are maintained while butterfat and milk protein levels are increased in dairy cows fed the instant feed compositions. It is important to note from the foregoing table that the daily milk fat yield was an average of 3.12 pounds per cow for the week beginning March 13, and the butterfat yield increased to 3.32 pounds per cow after feeding the Egg for four weeks. This 0.2 pound gain is a significant increase.

Using the April 2000 average butterfat value of $1.14/lb., feeding 0.25 pounds of Egg per day resulted in a $0.228 per cow per day financial benefit. At the same time, milk protein also increased, by 0.07 pounds per cow per day when Egg was fed. Milk protein in April 2000 was valued at $1.74/lb., which yielded an additional financial benefit of $0.122 per cow per day. Thus, feeding 0.25 pounds of Egg per day in this experimental herd was responsible for added income to the herdsman of $0.35 per cow per day.

In a second experiment employing the same methodology, similar improvements in milk fat production were seen. The herdsman reported that milk production remained approximately constant at 75 pounds per cow per day, while butterfat increased from 3.8% to 4.1%.

The skilled practitioner will recognize that these experimental results are unexpected and significant in light of prior efforts to increase butterfat and/or milk protein production. These results demonstrate that the present invention addresses a long-felt but previously unsolved need in the milk industry for an inexpensive, natural feed free from potentially harmful additives that can significantly increase butterfat and milk protein percentages, and thus increase the herdsman's income.

These improvements in butterfat and milk protein content are directly attributable to the nutritional profile of high quality inedible egg products according to the present invention. Fresh eggs are known to be a rich source of high quality protein. The egg white or albumen has a very high biological value in the amount and balance of amino acids. Egg albumen is frequently used as a reference to compare proteins from other sources when feeding animals such as the rat, mouse, chick, and others. The amino acid composition of whole hen's egg is used as the recommended profile for the Feed and Agriculture Organization's (1965) chemical score for required amino acids in protein (Galyean and Cotterill, 1995). High quality inedible egg products also contain various naturally occurring compounds with antimicrobial activity including, but not limited to, lysozyme that acts to hydrolyze β(1-4) glycosidic bonds in bacterial cell walls; ovotransferrin that acts to chelate Fe3+, Cu3+, Mn2+, Co2+, Cd2+, Zn2+, and Ni2+ to thereby render the minerals unavailable to bacteria until released by digestion of protein; avidin known to bind biotin thereby rendering it unavailable to bacteria until released by digestion of protein; ovoflavoprotein known to bind riboflavin thereby rendering it unavailable to bacteria until released by digestion of protein; and ovomucoid known to inhibit the action of various enzymes.

Prior efforts to increase butterfat and/or milk protein percentage have required the use of expensive, highly processed products that increase the herdsman's production costs and thereby diminish the return yielded by the higher market price for the improved butterfat and/or milk protein percentage milk. These prior efforts also have yielded only small improvements in butterfat and/or milk protein percentage and may introduce undesirable byproducts into the ruminant's milk.

The present invention avoids these drawbacks by providing a high quality, natural source of nutrition for far less cost and by improving both butterfat and milk protein percentages to a significant and unexpected degree. The present invention also provides a desirable amino acid profile for the ruminant that yields both healthier milk for the consumer and a healthier ruminant animal.

An additional benefit of the feed formulas of the present invention concerns the efficiency with which the instant formulas may be pelletized. The inedible egg fraction contained in the formula being pelleted serves as an excellent lubricant for the pelletizing apparatus. This makes the pelletizing equipment much more efficient than it is when pelletizing conventional dry feed formulations because it allows the pelletizing equipment to operate with a much lower power demand. This lower power demand decreases manufacturing costs for pelletized feed, and results again in a benefit to the herdsman of decreased feed costs for his or her animals. This also is an unexpected benefit of using high quality inedible egg products in a diet program for lactating ruminants.

Those of skill in the art will recognize that preferred diet programs incorporating feedstuff compositions according to the present invention are devised to meet the nutritional needs of the particular species being fed. Therefore, it is irrelevant what ingredients comprise the balance of feeds containing less than 100% high quality inedible egg, so long as those ingredients meet the nutritional requirements of the lactating ruminant for which the feed is intended. It is also irrelevant to the present invention when the skilled artisan decides to offer the present feed to the lactating ruminant, and it is irrelevant from which types of inedible egg the high quality inedible egg product is derived. What is relevant is that the various embodiments of the present invention are formulated to yield healthy lactating ruminants that produce milk with unexpected benefits for the herdsman, including unexpectedly high levels of butterfat and milk protein.

When lactating ruminants are fed the instant diet embodiments, whether or not the instant diet program is used, the antimicrobial compounds and nutritive components present in the liquid or dry inedible egg fraction minimize or eliminate the need to feed drugs to the animals, and thereby minimize or eliminate the significant costs presently associated with treating diseases in these animals. These compounds and nutritive components are also capable of curing certain microbe-caused diseases. Further, both the liquid and dry feedstuffs have an excellent amino acid profile, constitute rich sources of high quality protein, and are easy to digest. It is easy to pelletize compositions including a high quality inedible egg fraction because the egg lubricates the machinery and reduces power consumption of the pelletizing apparatus. Lactating ruminants fed compositions according to the present invention produce milk with increased levels of butterfat and milk protein over the levels of these milk components produced by animals fed a prior art feed composition.

While the invention has been described in detail in the foregoing description, the same is to be considered as illustrative and not restrictive in character, it being understood that only the preferred embodiments have been shown and described, and that all changes and modifications that come within the spirit of the invention are desired to be protected.

What is claimed is:

1. An egg-based feed composition for lactating ruminants, consisting essentially of, by weight:
high quality inedible egg from about 1% to 100%; and
at least one ingredient selected from the group consisting of:
milk product, cereal grain or cereal grain product, fruit pectin, other carbohydrate, fiber, fat, urea, electrolyte, vitamin, mineral, yeast, and other animal protein and/or vegetable protein balance.
